(19) United States
(12) Patent Application Publication
(10) Pub. No.: US 2026/0224262 A1
(43) Pub. Date: Aug. 6, 2026

(54) MEDICAL DEVICE INCLUDING COOLING SYSTEM, AND OPERATION METHOD THEREOF

(71) Applicant: WONTECH CO., LTD., Daejeon (KR)

(72) Inventors: Jong Won Kim, Seongnam (KR); Jung Hyun Kim, Seongnam (KR); Young Seok Seo, Sejong (KR); Ye Chan Nam, Uiwang (KR); Jung Il Park, Seongnam (KR)

(21) Appl. No.: 19/204,719

(22) Filed: May 12, 2025

(30) Foreign Application Priority Data
Feb. 6, 2025 (KR) 10-2025-0015003

(51) Int. Cl. A61B 18/00; A61B 90/00; G16H 40/63

(52) U.S. Cl. CPC A61B 18/00; A61B 90/06; G16H 40/63; A61B 2018/00005; A61B 2090/064

(57) ABSTRACT

Proposed are a medical device including a cooling system, and an operation method thereof, the medical device including a cooling can holder held with a cooling can mounted therein, a refrigerant chamber for accommodating a refrigerant, a refrigerant transfer unit for transferring the refrigerant toward a handpiece, one or more sensors for measuring pressure or refrigerant levels of the refrigerant chamber, and a control unit for outputting a gas discharge alarm or initiating a discharge operation of a gas discharge unit on the basis of measurement values, and it is effective in alleviating pain associated with a treatment and preventing thermal injury to a treatment area by transferring the refrigerant to the treatment area during skin treatment.

### CROSS REFERENCE TO RELATED APPLICATION

The present application claims priority to Korean Patent Application No. 10-2025-0015003, filed February 6, 2025, the entire contents of which are incorporated herein for all purposes by this reference.

### BACKGROUND OF THE INVENTION

### Field of the Invention

The present disclosure relates to a medical device including a cooling system and, more particularly, to a medical device capable of cooling a treatment area or a medical device during a medical treatment using high-frequency energy, ultrasound energy, laser energy, etc., and an operation method thereof.

### Description of the Related Art

Medical devices that perform skin treatments by using high-frequency energy, ultrasound energy, laser energy, etc. are being developed.

For example, representative medical devices for skin treatments include: laser devices for performing treatments such as skin toning and freckle removal by using Nd:YAG lasers or long pulse lasers; high-frequency devices for inducing collagen regeneration as well as skin restructuring in a dermal layer by using high-frequency electricity; and ultrasonic devices for improving skin elasticity and demonstrating a lifting effect by using ultrasound.

However, these medical devices utilizing such energy may cause problems of leading to pain during the treatments or causing thermal injuries due to fever at treatment areas. As a way to solve such problems, a method of cooling a treatment area may be effective. When the treatment area is cooled down, it is expected that a patient’s skin becomes less sensitive, making him or her feel much less pain, and that fever in the treatment area is alleviated, preventing thermal injury.

### SUMMARY OF THE INVENTION

A technical problem to be solved through exemplary embodiments of the present disclosure is to provide a medical device including a cooling system for transferring a refrigerant to a treatment area during skin treatment to alleviate pain accompanying the treatment and prevent thermal injury to the treatment area, and an operation method thereof.

Another technical problem to be solved through the exemplary embodiments of the present disclosure is to provide a medical device including a cooling system capable of resolving a problem of a refrigerant not being supplied smoothly due to excessive gas pressure in a refrigerant chamber, and an operation method thereof.

A yet another technical problem to be solved through the exemplary embodiments of the present disclosure is to provide a medical device including a cooling system capable of automatically detect a case of a cooling can that is not mounted or a case of an insufficient remaining amount of a refrigerant in the cooling can, so as to provide guidance such as on replacement of the cooling can, and an operation method thereof.

The technical problems of the present disclosure are not limited to the above-mentioned technical problems, and other technical problems not described above will be clearly understood by those skilled in the art from the description of the claims.

According to one exemplary embodiment of the present disclosure for solving the above-described problems, there is provided a medical device, including: a cooling can holder held with a cooling can mounted therein; a refrigerant chamber for accommodating a refrigerant introduced from the cooling can; a refrigerant transfer unit for transferring the refrigerant accommodated in the refrigerant chamber toward a handpiece; one or more sensors for measuring pressure or refrigerant levels of the refrigerant chamber; and a control unit for outputting a gas discharge alarm or initiating a discharge operation of a gas discharge unit on the basis of values obtained by measuring the pressure or refrigerant levels of the refrigerant chamber.

According to one exemplary embodiment of the present disclosure for solving the above-described problems, there is provided an operation method of a medical device, the operation method including: checking a first pressure value of a cooling can; checking a refrigerant level value of a refrigerant chamber when the first pressure value is greater than or equal to a first reference value and a second reference value; checking a second pressure value of the refrigerant chamber when the refrigerant level value is lower than a third reference value; and outputting a gas discharge alarm or initiating a discharge operation of a gas discharge unit when the refrigerant level value is lower than the third reference value and the second pressure value is higher than a fourth reference value.

According to the above-described exemplary embodiments of the present disclosure, a refrigerant is transferred to a treatment area during skin treatment, thereby being capable of alleviating pain accompanying the treatment and preventing thermal injury to the treatment area.

In addition, a cooling system is configured in an embedded form in a medical device, whereby the entire system becomes simpler and is not required to have a separate cooling system from the medical device.

In addition, a means for effectively detecting and discharging gas pressure when the gas pressure inside a refrigerant chamber becomes excessively high is provided, whereby the problem of a refrigerant not being supplied smoothly due to the excessive gas pressure inside the refrigerant chamber may be resolved.

In addition, in a case of a cooling can that is not mounted or a case of an insufficient remaining amount of a refrigerant in the cooling can, this case is automatically detected and guidance such as on replacement of the cooling can is provided, thereby enabling a user to easily manage the cooling can.

In addition, a pressure drop in a cooling can may be compensated for through a cooling-can heater, and a time and interval of heating is controlled, whereby a problem of the cooling can exploding due to excessive heating may be prevented.

In addition, in a case where gas pressure inside a refrigerant chamber rapidly increases, the gas pressure is forcibly discharged through a high pressure protector capable of operating independently of a gas discharge unit, whereby a problem such as damage to a refrigerant chamber due to the excessive gas pressure may be prevented.

The technical effectiveness of the present disclosure are not limited to the above-mentioned effectiveness, and other technical effectiveness not described above will be clearly understood by those skilled in the art from the description of the claims.

### BRIEF DESCRIPTION OF THE DRAWINGS

FIGS. 1A and 1B are views illustrating a medical device including a cooling system according to an exemplary embodiment of the present disclosure.

FIG. 2 is a view illustrating main components of the cooling system according to the exemplary embodiment of the present disclosure.

FIG. 3 is a view additionally illustrating detailed components of a refrigerant chamber 150 and a level sensor 170 illustrated in FIG. 2.

FIGS. 4A, 4B, and 5 are views specifically illustrating not only a problem when pressure inside the refrigerant chamber 150 increases but also a gas discharge method in this case.

FIG. 6 is a flowchart illustrating an operation method for managing a cooling can of the medical device according to one exemplary embodiment of the present disclosure.

FIG. 7 is a flowchart illustrating an operation method for managing pressure in the refrigerant chamber of the medical device according to the exemplary embodiments of the present disclosure.

FIG. 8 is a flowchart illustrating one exemplary embodiment that further concretizes step S270 in FIG. 7.

FIG. 9 is a flowchart illustrating an overall operation method related to a cooling function of the medical device according to the exemplary embodiments of the present disclosure.

FIG. 10 is a flowchart illustrating an exemplary computing device in which an operation method of a medical device is implemented according to the exemplary embodiments of the present disclosure.

### DETAILED DESCRIPTION OF THE INVENTION

Hereinafter, exemplary embodiments of the present disclosure are described with reference to the accompanying drawings. Advantages and features of the present disclosure and the methods of achieving the same will become apparent with reference to the exemplary embodiments described below in detail in conjunction with the accompanying drawings. However, the technical ideas of the present disclosure is not limited to the exemplary embodiments disclosed below, but will be implemented in a variety of different forms. These exemplary embodiments are provided only to complete the technical ideas of the present disclosure and to completely inform the scope of the present disclosure to those skilled in the art to which the present disclosure pertains, and the technical ideas of present disclosure are only defined by the scope of the claims.

In adding reference numerals to the components of each drawing, it should be noted that the same reference numerals are used for referring to the same components as much as possible even if displayed on different drawings. In the following description of the present disclosure, detailed descriptions of related known functions and components incorporated herein will be omitted when it is determined that the subject matter of the present disclosure may be obscured thereby.

Unless otherwise defined, all terms (including technical and scientific terms) used in the present description may be used in a sense that may be commonly understood by those skilled in the art. In addition, terms defined in the commonly used dictionary are not ideally or excessively interpreted unless explicitly and specifically defined otherwise. The terminology used herein is for the purpose of describing particular exemplary embodiments only and is not intended to be limiting of the embodiments of the present disclosure. In the present specification, the singular form also includes the plural form unless otherwise specified in the phrase.

Further, when describing the components of the present disclosure, terms such as first, second, A, B, (a) or (b) may be used. Since these terms are provided merely for the purpose of distinguishing the components from each other, they do not limit the nature, sequence, or order of the components. When a component is described as being “connected”, “coupled”, or “linked” to another component, that component may be directly connected or linked to that other component. However, it should be understood that yet another component between each of the components may be “connected”, “coupled”, or “linked” to each other.

Hereinafter, some exemplary embodiments of the present disclosure will be described in detail with reference to the attached drawings.

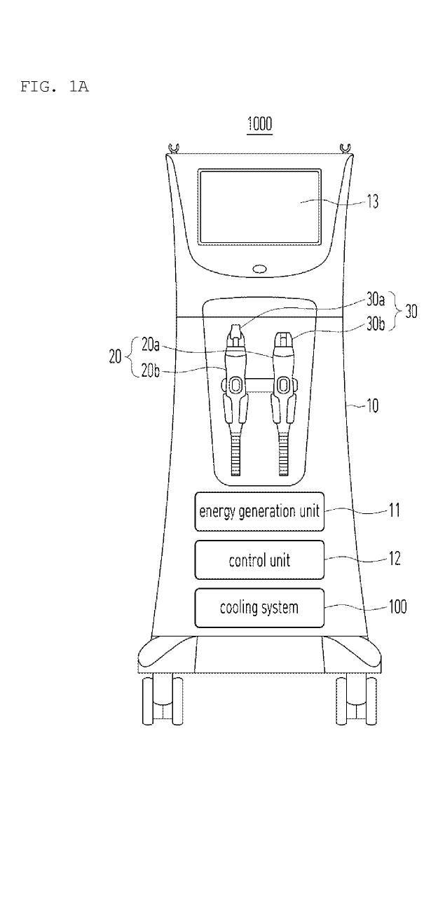

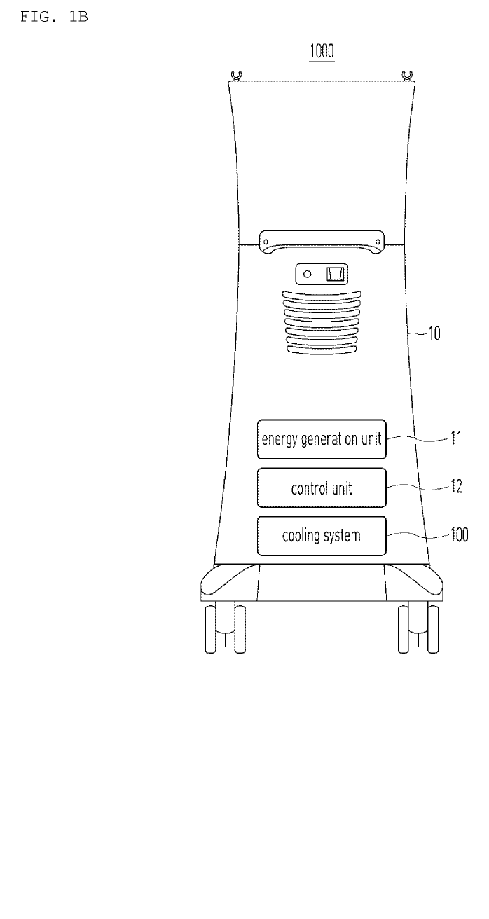

FIGS. 1A and 1B are views illustrating a medical device including a cooling system according to an exemplary embodiment of the present disclosure. FIG. 1A illustrates the front side of a medical device 1000, and FIG. 1B illustrates the rear side of the medical device 1000.

Referring to FIGS. 1A and 1B, the medical device according to the exemplary embodiment of the present disclosure may include a main body 10, a handpiece 20, and a tip 30.

The main body 10 is configured to generate energy for skin treatment, transmit the generated energy to the handpiece 20, and provide a user interface for overall control of the medical device 1000. To this end, the main body 10 may be provided with an energy generation unit 11, a control unit 12, and a display 13.

The energy generation unit 11 may generate different kinds of energy depending on types of medical devices 1000. For example, in a case of a medical device 1000 that is a high-frequency device, an energy generating unit 11 may generate electrical energy. Alternatively, in a case of a medical device 1000 that is a laser device, an energy generating unit 11 may generate laser light.

The control unit 12 controls the overall operation of the components 11, 13, and 100 of the main body 10. For example, the control unit 12 may initiate or terminate an energy-generating operation of the energy generation unit 11, a user interface-displaying operation of the display 13, and/or a cooling operation of the cooling system 100.

The display 13 is a component for displaying a user interface and may be an electronic display means such as a Liquid Crystal Display (LCD), a Light Emitting Diode (LED), or an Organic Emitting Diode (OLED).

Meanwhile, the main body 10 supplies a refrigerant for cooling a treatment area during skin treatment, and may also supply a refrigerant for the purpose of cooling the handpiece 20 and tip 30 that are becoming overheated during a treatment process. To this end, the main body 10 may further be provided with a cooling system 100.

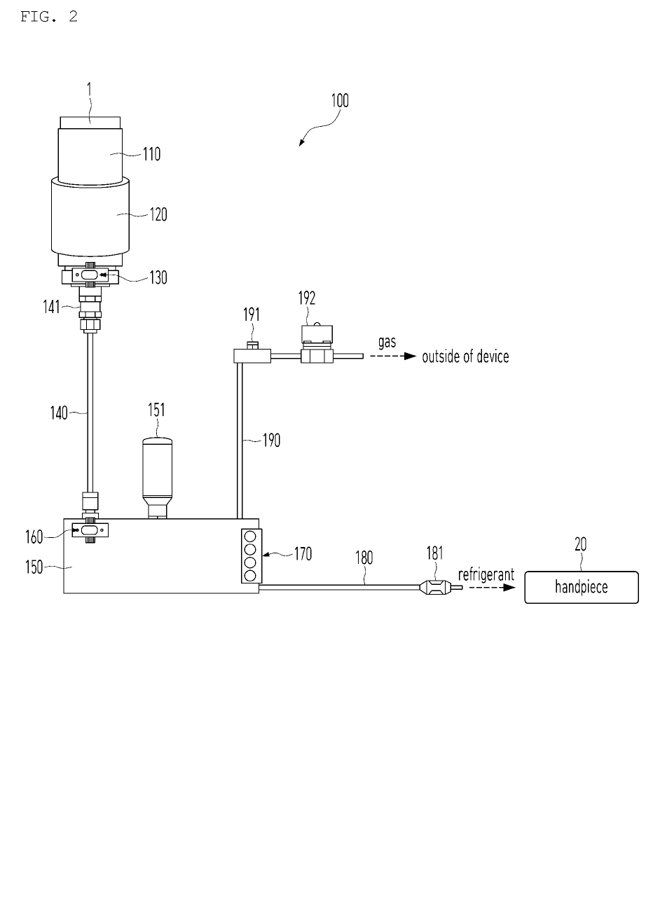

The specific components and operation of the cooling system 100 will be described in detail later in FIG. 2 and below.

The handpiece 20 is a component for receiving transmitted energy generated by the energy generating unit 11 and transmitting this energy to the tip 30. The tip 30 is a component for outputting the energy transmitted from the handpiece 20 toward the skin of a user.

The handpiece 20 and tip 30 may have different types depending on the types of medical devices 1000. For example, in a case of a medical device 1000 that is an ultrasonic medical device, a handpiece 20 and tip 30 serve as a handpiece and tip for ultrasonic output. In this case, the tip 30 may be provided with a transducer for converting the transmitted energy into ultrasonic waves. As another example, in a case of a medical device 1000 that is a high-frequency medical device, a handpiece 20 and tip 30 serve as a handpiece and tip for high-frequency output. In this case, the tip 30 may be provided with one or more electrodes for emitting high frequency waves to the skin of a user.

As the exemplary embodiment, as illustrated in FIG. 1, two or more handpieces 20 and tips 30 may be provided. In this case, each of the handpieces 20a and 20b and each of the tips 30a and 30b may be a different type of handpiece and tip. For example, a first handpiece 20a and a first tip 30a may be a handpiece and tip for outputting ultrasonic waves, and a second handpiece 20b and a second tip 30b may be a handpiece and tip for outputting high-frequency waves. As another example, a first handpiece 20a and a first tip 30a may respectively be an invasive handpiece and an invasive tip for transmitting high-frequency waves in an invasive manner, and a second handpiece 20b and a second tip 30b may respectively be a non-invasive handpiece and a non-invasive tip for transmitting high-frequency waves in a non-invasive manner.

FIG. 2 is a view illustrating main components of a cooling system according to the exemplary embodiment of the present disclosure. Referring to FIG. 2, the cooling system 100 may include a cooling can holder 110, a heater 120, a refrigerant chamber 150, a water hammer arrestor 151, a refrigerant transfer unit 181, a high pressure protector 191, a gas discharge unit 192, and/or one or more sensors 130, 160, and 170. In addition, the cooling system 100 may further include a first valve 141, a first flow path 140, a second flow path 180, and/or a third flow path 190, which are for movement of a refrigerant.

The cooling can 1 is mounted in the cooling can holder 110. The cooling can 1 accommodates the refrigerant in a fluid state inside a metal casing and supplies the refrigerant for the operation of the cooling system 100.

The cooling can holder 110 may have a cylindrical shape as illustrated in FIG. 2 to stably accommodate the cooling can 1, but is not limited thereto. For example, the cooling can holder 110 may have various shapes, such as a disk shape, a pipe shape, or a tongs shape.

The heater 120 is a component for heating the cooling can 1 mounted in the cooling can holder 110. For example, in a case where pressure inside the cooling can 1 is too low, the heater 120 may heat the cooling can 1 so as to increase the pressure inside the cooling can 1. This may help further facilitate transfer of a refrigerant to the refrigerant chamber 150.

For example, in a case where pressure inside the cooling can 1 is lower than pressure in the refrigerant chamber 150, it is difficult for the refrigerant in the cooling can 1 to be transferred to the refrigerant chamber 150 due to the characteristic of a fluid flowing from a place of high pressure to a place of low pressure. In this case, when the cooling can 1 is heated through the heater 120, the pressure of the cooling can 1 may become higher than the pressure of the refrigerant chamber 150, and the transfer of the refrigerant may be made more easily.

As one exemplary embodiment, the heater 120 may have an annular or curved shape that at least partially surrounds the cooling can 1 or the cooling can holder 110.

The first pressure sensor 130 is a sensor for measuring the pressure of a cooling can 1. A pressure value that is of the cooling can 1 and measured by the first pressure sensor 130 (hereinafter referred to as a “first pressure value”) may be used for determining the presence or absence of the cooling can 1. For example, in a case where the first pressure value measured by the first pressure sensor 130 is lower than a first reference value (or, a NoCan reference value), the control unit 12 may determine that the cooling can 1 is not mounted in the cooling can holder 110, or that the refrigerant is depleted even though the cooling can 1 is mounted. In this case, the control unit 12 may output an alarm for mounting or replacing the cooling can 1. The alarm may be an alarm indicating that the cooling can is not mounted in the cooling can holder.

As one exemplary embodiment, the control unit 12 may output the alarm in a way of displaying the alarm on the display 13 or providing voice guidance through the speaker provided in the main body 10.

In addition, the first pressure value may be used for controlling the operation of the heater 120. For example, the first pressure value measured by the first pressure sensor 130 is transmitted to the control unit 12, and then the control unit 12 may operate the heater 120 to increase the pressure of the cooling can 1 in a case where the first pressure value is lower than a heating reference value (hereinafter referred to as a “second reference value”). This may facilitate smoother transfer of a refrigerant from the cooling can 1 to the refrigerant chamber 150.

As one exemplary embodiment, the control unit 12 may monitor pressure (e.g., a first pressure value) of the cooling can 1 or a temperature of the cooling can 1, and operate the heater 120 until the pressure of the cooling can 1 reaches a predetermined pressure value, or until the temperature of the cooling can 1 reaches a predetermined temperature value.

As one exemplary embodiment, when the heater 120 of the control unit 12 is operated, the operation of the heater 120 may be controlled so that operation sections of the heater 120 include two or more heating sections and one or more stop sections. This may facilitate heating the cooling can 1 more safely.

For example, when the heater 120 is operated for heating continuously until certain pressure or a certain temperature is reached, the cooling can 1 may be heated too rapidly by mistake, thereby posing a risk of causing an explosion of the cooling can 1. To avoid such a risk, the control unit 12 may control the heater 120 so that when the heater 120 is operated, a heating section (i.e., a section where the heater operates for heating) is repeated in a predetermined cycle, and a regular section (i.e., a section where the heater does not operate for heating) is arranged between heating sections. This facilitates to maximally reduce the risk of causing the explosion of the cooling can 1 by allowing the cooling can 1 to heat up gradually.

Meanwhile, when the cooling can 1 is mounted in the cooling can holder 110, the first valve 141 is opened and the refrigerant of the cooling can 1 is transferred to the refrigerant chamber 150 through the first flow path 140.

As one exemplary embodiment, the opening and closing of the first valve 141 may be electronically controlled by the control unit 12, and in this case, the first valve 141 may be a solenoid valve.

The refrigerant chamber 150 accommodates and stores the refrigerant introduced from the cooling can 1. The refrigerant stored in the refrigerant chamber 150 may be transferred to the handpiece 20 through the second flow path 180 and the refrigerant transfer unit 181. The refrigerant transferred to the handpiece 20 may be applied to a treatment area or used for cooling the components of the handpiece 20 or tip 30.

As one exemplary embodiment, the refrigerant transfer unit 181 may include a second valve (not shown) and the control unit 12 may control the second valve, thereby allowing or blocking the transfer of refrigerant to the handpiece 20.

Meanwhile, one or more sensors 160 and 170 may be provided in order to measure pressure and refrigerant levels of the refrigerant chamber 150.

A second pressure sensor 160 measures the pressure of the refrigerant chamber 150. In this case, a pressure value measured by the second pressure sensor 160 (hereinafter referred to as the “second pressure value”) may represent gas pressure inside the refrigerant chamber 150.

For example, in a case where a refrigerant is stored in a gaseous state within the refrigerant chamber 150, a second pressure value may directly represent the pressure of the refrigerant in the gaseous state. As another example, in a case where a refrigerant is stored in a liquid state within the refrigerant chamber 150, a second pressure value may represent gas pressure of a portion that is not filled with the refrigerant.

The level sensor 170 measures levels of refrigerant stored in the refrigerant chamber 150. Accordingly, the level sensor 170 may be a component configured to function effectively when the refrigerant is stored in the liquid state.

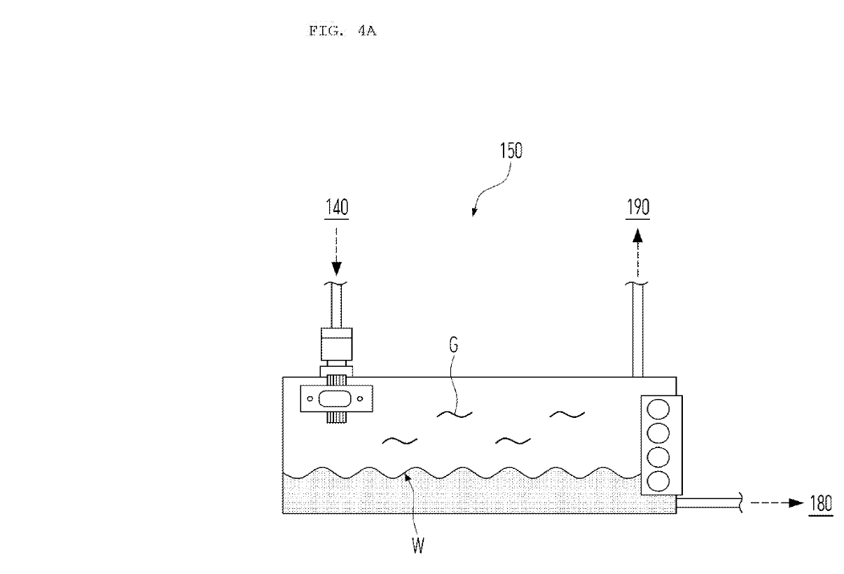

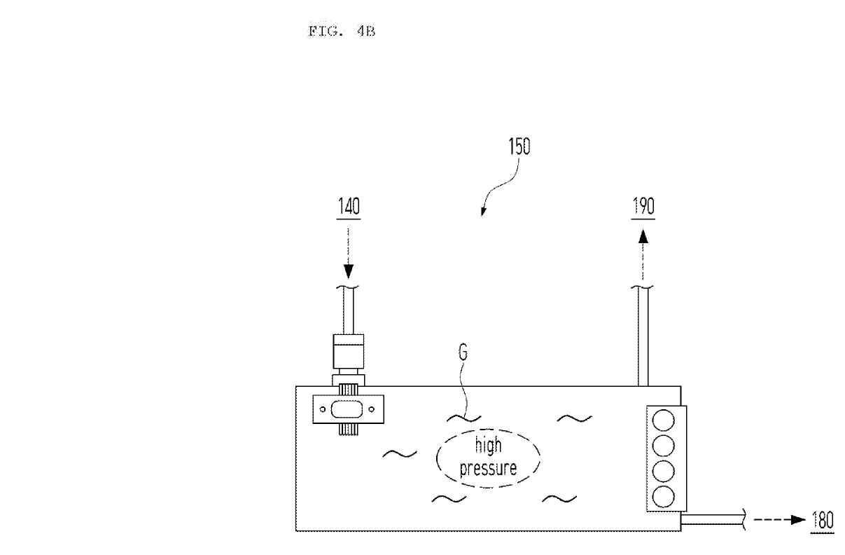

The second pressure value measured by the second pressure sensor 160 and/or the refrigerant level value measured by the level sensor 170 may be used for controlling gas discharge from the refrigerant chamber 150. For example, in a case where the gas pressure inside the refrigerant chamber 150 is high, refrigerant inflow from the cooling can 1 may become difficult. In particular, in a case where the gas pressure becomes excessively high, there may be a risk of causing an explosion of the refrigerant chamber 150. In this case, an operation is required to discharge the gas inside the refrigerant chamber 150 to the outside and lower the gas pressure inside the refrigerant chamber 150. The second pressure value and/or the refrigerant level value may be used for controlling this operation. This will be described in more detail in FIGS. 4A and 4B and below.

The water hammer arrestor 151 is a component for mitigating a water hammer phenomenon caused by the flow of refrigerant. The water hammer arrestor 151 may be coupled to a side surface of the refrigerant chamber 150.

As one exemplary embodiment, the water hammer arrestor 151 is provided with a spring inside thereof and may absorb the shock applied to the refrigerant chamber 150 due to the flow of refrigerant through the elastic energy of the spring.

The gas discharge unit 192 is a component for discharging gas from the refrigerant chamber 150 to the outside of the device. The gas discharge unit 192 may include a third valve (not shown).

As one exemplary embodiment, the third valve may be a solenoid valve. The opening and closing of the third valve may be electronically controlled by the control unit 12. When the third valve is opened by the control unit 12, the gas inside the refrigerant chamber 150 is transferred to the gas discharge unit 192 via the third flow path 190 and discharged to the outside of the device through the opened third valve. In contrast, when the third valve is closed by the control unit 12, the gas discharge from the refrigerant chamber 150 is also blocked.

The high pressure protector 191 is a component for preventing an explosion due to excessively high gas pressure inside the refrigerant chamber 150. When the gas pressure inside the refrigerant chamber 150 becomes greater than or equal to a certain level, the high pressure protector 191 is destroyed and the gas inside the refrigerant chamber 150 is discharged through a destroyed part. For example, the high pressure protector 191 may function as a fuse that limits an upper limit of the gas pressure inside the refrigerant chamber 150.

The high pressure protector 191 is provided for safety reasons and configured so as to forcibly discharge the gas inside the refrigerant chamber 150 even in a state where the discharge operation of the gas discharge unit 192 has not been initiated. To this end, the high pressure protector 191 may be installed on the path of the third flow path 190.

According to the exemplary embodiment described with reference to FIG. 2, there is provided the cooling system for transferring the refrigerant from the cooling can 1 to the handpiece 20. The refrigerant provided by the handpiece 20 is provided to a treatment area, so as to alleviate pain of a patient and prevent thermal injury to the treatment area.

In addition, since the cooling system provides a means for discharging gas pressure inside the refrigerant chamber 150 to the outside when the gas pressure becomes excessive, the problem of the refrigerant not being supplied smoothly due to the gas pressure inside the refrigerant chamber 150 may be resolved.

In addition, a case where the cooling can 1 is not mounted or the remaining amount of the refrigerant in the cooling can 1 is insufficient is automatically detected, so as to provide the guidance on replacing the cooling can 1, thereby enabling a user to manage the cooling can easily.

In addition, a pressure drop in the cooling can 1 may be compensated for through the heater 120, and the problem with the cooling can 1 exploding due to excessive heating may be prevented by controlling a time and interval of heating,

In addition, a case where gas pressure inside the refrigerant chamber 150 becomes excessively high, the gas pressure is forcibly discharged through the high-pressure protector 191 capable of operating independently of the gas discharge unit 192, so that the problems such as the refrigerant chamber 150 exploding due to the gas pressure may be prevented in advance.

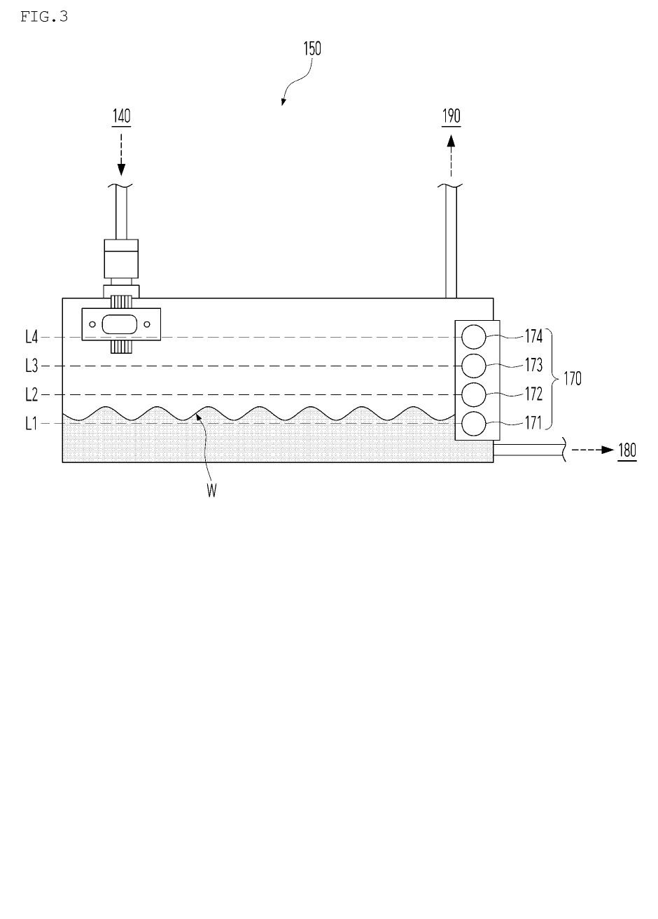

FIG. 3 is a view additionally illustrating detailed components of the refrigerant chamber 150 and level sensor 170 illustrated in FIG. 2. In FIG. 3, one exemplary embodiment of measuring a refrigerant level in the refrigerant chamber 150 by using the level sensor 170 is described.

Referring to FIG. 3, the level sensor 170 is composed of one or more level sensors 171, 172, 173, and 174, and the one or more level sensors 171, 172, 173, and 174 correspond to respective refrigerant levels different from each other in the refrigerant chamber 150. For example, a first level sensor 171 may correspond to a first refrigerant level L1, a second level sensor 172 may correspond to a second refrigerant level L2, a third level sensor 173 may correspond to a third refrigerant level L3, and a fourth level sensor 174 may correspond to a fourth refrigerant level L4.

As one exemplary embodiment, each of the one or more level sensors 171, 172, 173, and 174 may detect, as a binary value, whether a refrigerant exists or not at a corresponding refrigerant level L1, L2, L3, or L4. For example, as illustrated in FIG. 3, a case where a water surface W of the refrigerant is positioned between the first refrigerant level L1 and the second refrigerant level L2 means that the refrigerant exists at the first refrigerant level L1, so the first level sensor 171 outputs a measurement value of “on”. In contrast, the refrigerant is not present in the refrigerant levels L2, L3, and L4 other than that level, so all the second level sensor 172, the third level sensor 173, and the fourth level sensor 174 output respective measurement values of “off”.

The measurement values of the one or more level sensors 171, 172, 173, and 174 are transmitted to the control unit 12, and the control unit 12 determines a refrigerant level of the refrigerant chamber 150 on the basis of the measurement values. At this time, the control unit 12 may determine, as a current refrigerant level value of the refrigerant chamber 150, any one of values that are greater than or equal to a level confirmed as “on” and less than a level confirmed as “off”. For example, in the example in FIG. 3, the control unit 12 may determine the first refrigerant level L1 as the current refrigerant level value.

As one exemplary embodiment, in a case where all the measurement values of the one or more level sensors 171, 172, 173, and 174 are “off”, the control unit 12 may determine that the current refrigerant level value is 0 or below a reference value.

Meanwhile, the control unit 12 may display a corresponding remaining amount of the refrigerant on the display 13 on the basis of the refrigerant level values, which are of the refrigerant chamber 150 and measured by the one or more level sensors 171, 172, 173, and 174.

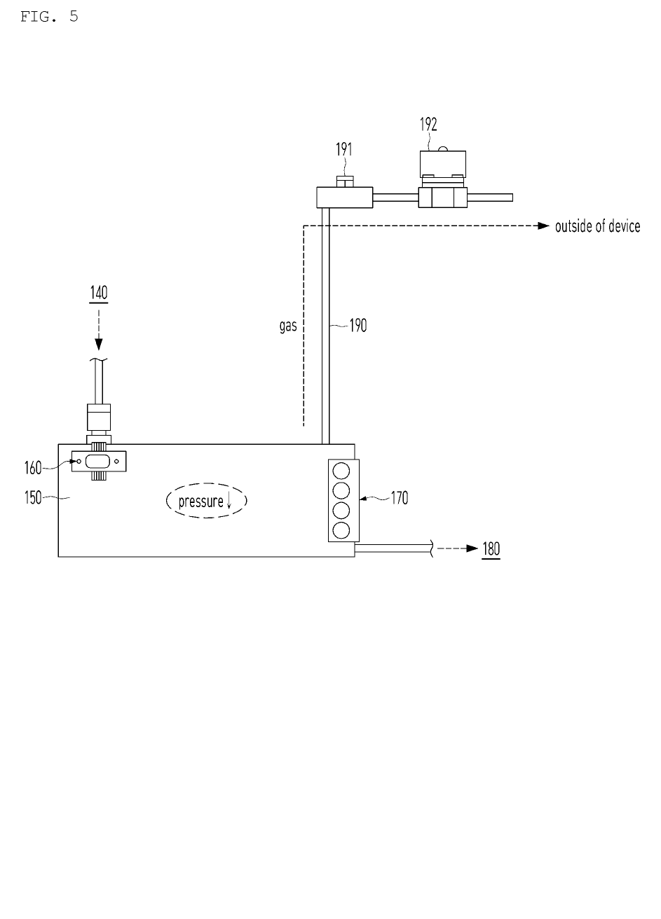

FIGS. 4A, 4B, and 5 are views specifically illustrating not only a problem occurring when pressure inside the refrigerant chamber 150 increases but also a gas discharge method for this case. FIG. 4A is a view illustrating the refrigerant chamber 150 when gas pressure G is within a normal range, and FIG. 4B is a view illustrating the refrigerant chamber 150 when gas pressure G becomes excessively high.

In the example in FIG. 4A, since the gas pressure G is within the normal range, the refrigerant in the cooling can 1 may be introduced through the first flow path 140, and the refrigerant chamber 150 is filled with an appropriate water level W of the refrigerant. The refrigerant in the refrigerant chamber 150 may be transferred to the handpiece 20 via the second flow path 180 under the control of the control unit 12 and be used for cooling a treatment area, etc.

In the example in FIG. 4B, since the gas pressure G is in a high state, the pressure of the refrigerant chamber 150 becomes excessively higher than the pressure of the cooling can 1, making it difficult for the refrigerant in the cooling can 1 to flow in. Accordingly, the refrigerant in the refrigerant chamber 150 is gradually depleted, and the refrigerant required for cooling is unable to be transferred to the handpiece 20.

Accordingly, in this case, it is required to discharge the gas inside the refrigerant chamber 150, so as to lower the gas pressure G. A detailed description will be continued with reference to FIG. 5.

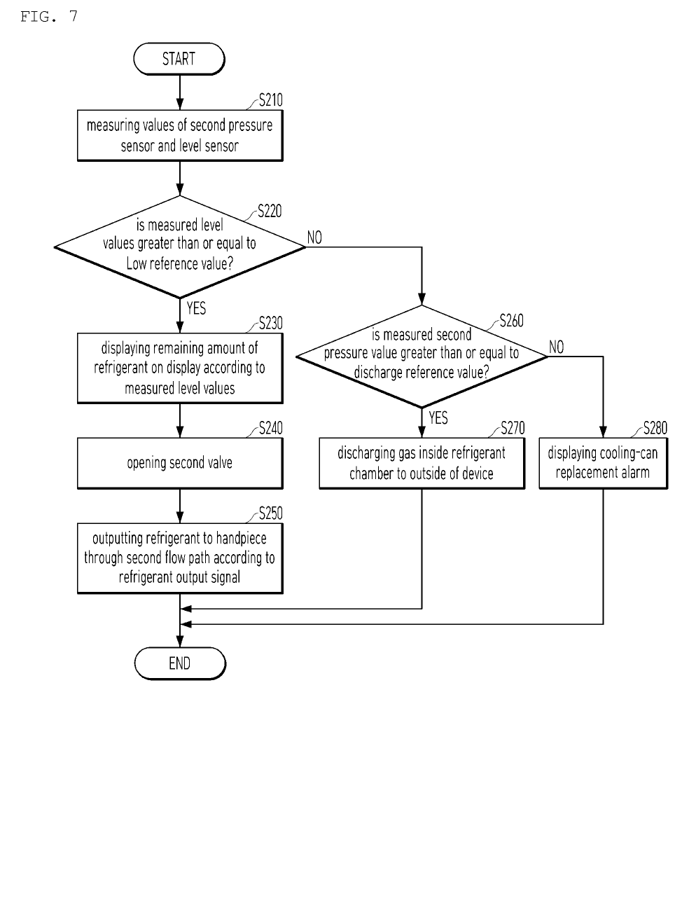

Referring to FIG. 5, the control unit 12 measures pressure (i.e., the gas pressure) and a refrigerant level value of the refrigerant chamber 150 through the second pressure sensor 160 and/or the level sensor 170. In addition, based on the measured value, it is determined whether a gas discharge condition of the refrigerant chamber 150 has been met. A specific example of the gas discharge condition of the refrigerant chamber 150 is described in detail in FIG. 7 and below, so a description thereof is omitted here.

When the gas discharge condition of the refrigerant chamber 150 is met, the control unit 12 may output a gas discharge alarm, thereby enabling a user to directly initiate a gas discharge operation, or controlling the gas discharge unit 192 to automatically initiate the gas discharge operation of the gas discharge unit 192. Here, for the sake of simplicity of description, the description will be given assuming a case where the control unit 12 automatically initiates the gas discharge operation of the gas discharge unit 192.

The control unit 12 controls the gas discharge unit 192 so that the third valve is opened when the gas discharge condition of the refrigerant chamber 150 is met. As the third valve is opened, the gas inside the refrigerant chamber 150 reaches the gas discharge unit 192 via the third flow path 190 and is discharged outside the device. As the gas inside the refrigerant chamber 150 is discharged to the outside, the gas pressure inside the refrigerant chamber 150 gradually decreases. When the gas pressure reaches the normal range, the refrigerant in the cooling can 1 is introduced through the first flow path 140, so the refrigerant is filled in the refrigerant chamber 150.

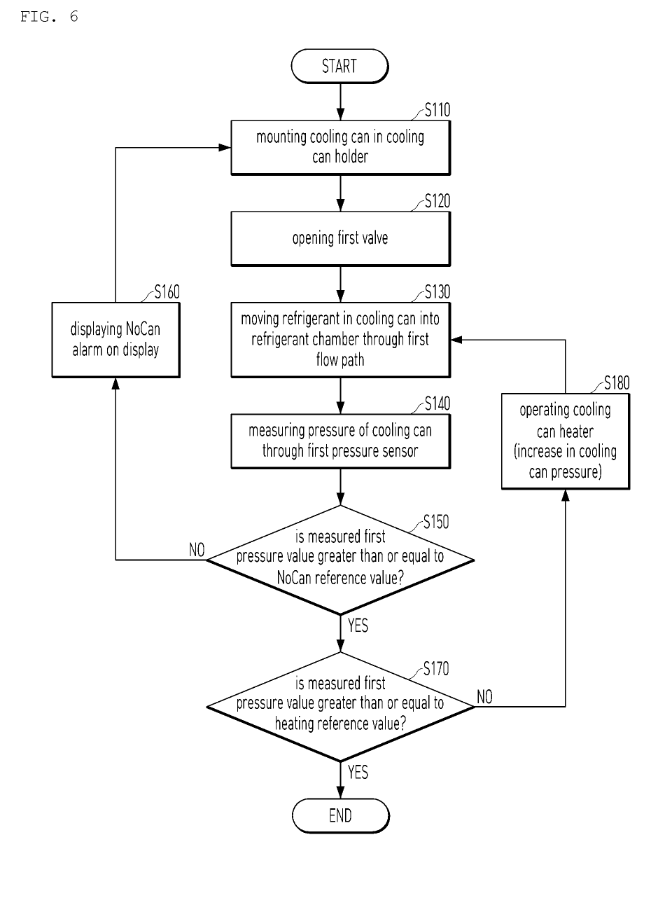

FIG. 6 is a flowchart illustrating an operation method for managing a cooling can of a medical device according to an exemplary embodiment of the present disclosure. The operation method described in the exemplary embodiment in FIG. 6 may be performed by the control unit 12 in FIG. 1. Accordingly, when a performing subject is omitted in steps below, it is assumed that the performing subject is the control unit 12. In the description of the present exemplary embodiments, as for the same content as the content described above in FIGS. 1 to 5, the description thereof may be omitted in order to avoid the duplication of description.

In step S110, a cooling can is mounted in a cooling can holder.

In step S120, a first valve is opened. The opening or closing of the first valve may be electronically controlled by a control unit 12.

In step S130, a refrigerant in the cooling can moves into a refrigerant chamber through a first flow path.

In step S140, a first pressure value of the cooling can is measured through a first pressure sensor.

In step S150, it is checked whether the first pressure value is greater than or equal to a first reference value (e.g., a NoCan reference value). Here, the first reference value means a preset reference value for determining whether the cooling can is mounted or whether the refrigerant in the cooling can is depleted.

When the first pressure value is less than the first reference value, this means that the cooling can is not mounted or the refrigerant in the cooling can is depleted, so the present exemplary embodiment proceeds to step S160 and outputs a cooling-can replacement alarm (i.e., a NoCan alarm) through a display or a speaker so as to enable a user to replace the cooling can.

When the first pressure value is greater than or equal to the first reference value, the present exemplary embodiment proceeds to step S170.

In step S170, it is checked whether the first pressure value is greater than or equal to a second reference value (e.g., a heating reference value). Here, the second reference value means a preset reference value for triggering the operation of a heater.

When the first pressure value is less than the second reference value, this means that the pressure of the cooling can is low, so the present exemplary embodiment proceeds to step S180, and the heater is operated to heat the cooling can, thereby increasing the pressure of the cooling can.

When the first pressure value is greater than or equal to the second reference value, it means that the pressure of the cooling can is within an appropriate range, so the present exemplary embodiment is terminated without any additional control.

FIG. 7 is a flowchart illustrating an operation method for managing pressure in the refrigerant chamber of the medical device according to the exemplary embodiment of the present disclosure. The operation method described in one exemplary embodiment in FIG. 7 may be performed by the control unit 12 in FIG. 1. Accordingly, in a case where a performing subject is omitted in steps below, it is assumed that the performing subject is the control unit 12. In the description of the present exemplary embodiments, as for the same content as the content described above in FIGS. 1 to 5, the description thereof may be omitted in order to avoid the duplication of description.

In step S210, a second pressure value of a second pressure sensor and/or a refrigerant level value of a level sensor are measured.

In step S220, it is checked whether the refrigerant level value (or the level value) is greater than or equal to a third reference value (e.g., a Low reference value).

Here, the third reference value is a preset reference level value for determining a gas discharge condition of the refrigerant chamber, and may be, for example, the first refrigerant level L1 in FIG. 3.

When the refrigerant level value is greater than or equal to the third reference value, this means that a certain amount or more of the refrigerant exists in the cooling chamber, so the present exemplary embodiment proceeds to step S230.

In step S230, the remaining amount of the refrigerant corresponding to the refrigerant level value is displayed on the display on the basis of the refrigerant level value.

In step S240, the handpiece is operated to perform a skin treatment procedure, and a second valve is opened to cool a treatment area or the handpiece, etc.

In step S250, the refrigerant is output to the handpiece through a second flow path according to a refrigerant output signal that is input from the control unit or the handpiece.

Meanwhile, referring back to step S220, when the refrigerant level value is less than the third reference value, this means that the refrigerant in the cooling chamber is depleted, so the present exemplary embodiment proceeds to step S260 in order to determine whether the gas discharge condition is met.

In step S260, it is checked whether the second pressure value is greater than or equal to a fourth reference value (e.g., a discharge reference value).

Here, the fourth reference value means a preset reference pressure value for determining the gas discharge condition of the refrigerant chamber.

When the second pressure value is greater than or equal to the fourth reference value, it may be assumed that the gas pressure inside the refrigerant chamber is excessively high and the refrigerant is not introduced into the refrigerant chamber. In this case, the present exemplary embodiment proceeds to step S270, and under the control of the control unit, the gas inside the refrigerant chamber is discharged to the outside of the device, or a gas discharge alarm is output so as to enable the user to directly initiate a gas discharge operation.

When the second pressure value is less than the fourth reference value, a cause of the refrigerant depletion in the refrigerant chamber is not due to gas pressure. In this case, it may be assumed that the cause of the refrigerant depletion is due to an insufficient remaining amount of refrigerant in the cooling can. Accordingly, the present exemplary embodiment proceeds to step S280, and a refrigerant shortage alarm or a cooling-can replacement alarm may be output through the display or speaker so as to enable the user to replace the cooling can.

Meanwhile, in the exemplary embodiment in FIG. 7, as the gas discharge condition of the refrigerant chamber, a case is exemplified requiring the following items: i) a refrigerant level value is less than the third reference value, and ii) a second pressure value is greater than or equal to the fourth reference value. However, the scope of the present disclosure is not limited thereto.

For example, the gas discharge condition of the refrigerant chamber may include only one item that the second pressure value is greater than or equal to the fourth reference value. That is, when the second pressure value is higher than a certain level, it is determined that there may be an obstacle to the inflow of refrigerant, and gas discharge may be initiated regardless of the refrigerant level value.

In this case, the control unit receives the second pressure value from the second pressure sensor, and may output the gas discharge alarm or initiate the discharge operation of the gas discharge unit when the second pressure value is higher than the fourth reference value.

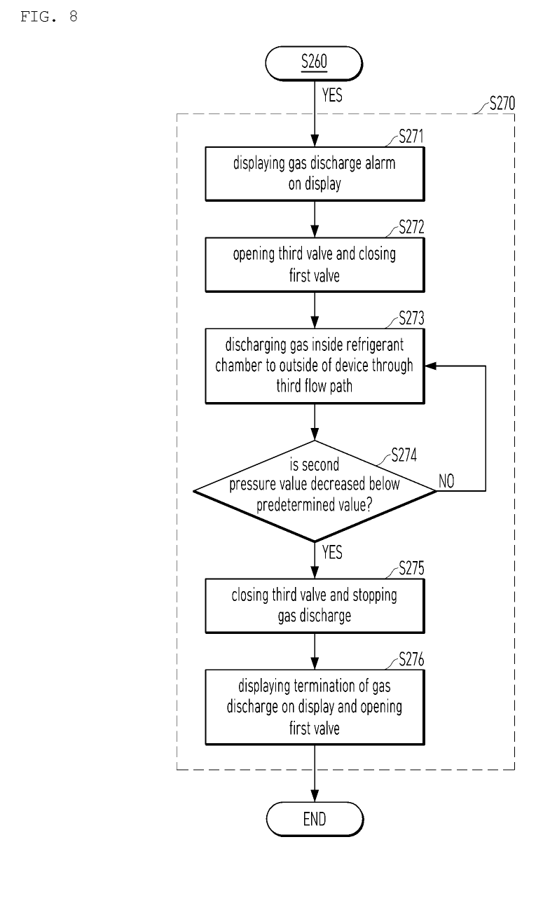

FIG. 8 is a flowchart illustrating one exemplary embodiment that further concretizes step S270 in FIG. 7.

In step S271, the gas discharge alarm is displayed on the display.

In step S272, a third valve is opened for gas discharge. At this time, the first valve may be closed in order to prevent the newly introduced refrigerant out of the cooling can from vaporizing and from being discharged together through the third valve.

In step S273, the gas inside the refrigerant chamber is discharged outside the device through a third flow path.

In step S274, it is checked whether a second pressure value is decreased to a value less than a predetermined value (e.g., a fourth reference value).

When the second pressure value is not decreased to the value less than the predetermined value, this means that the gas pressure in the refrigerant chamber is not sufficiently lowered, so the process returns to step S273 and the gas discharge continues.

When the second pressure value is decreased to the value less than the predetermined value, this means that the gas pressure in the refrigerant chamber is sufficiently lowered, so the process proceeds to step S275, thereby closing the third valve and stopping the gas discharge accordingly.

In step S276, the display shows that the gas discharge is terminated, and the first valve is opened again in order to supply a refrigerant from the cooling can.

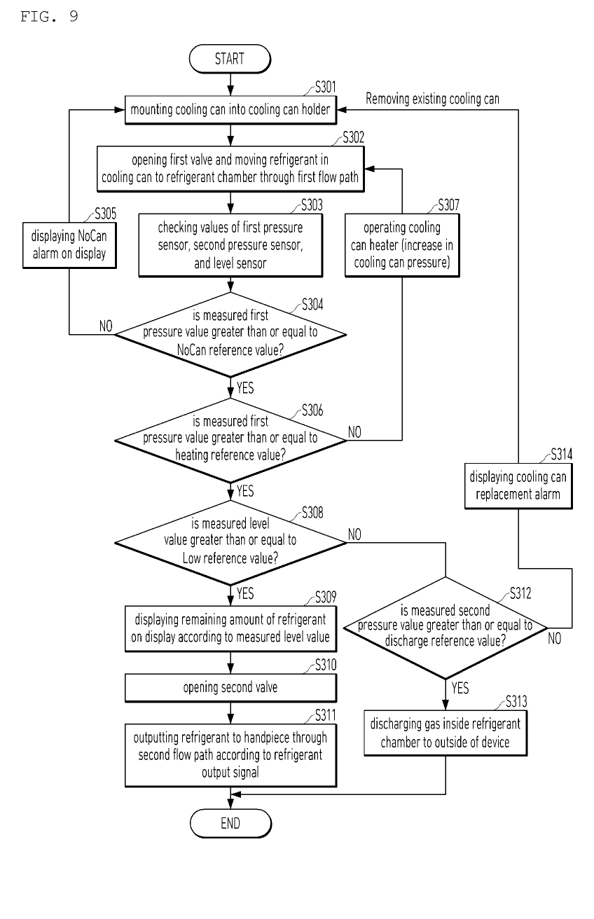

FIG. 9 is a flowchart illustrating an overall operation method related to a cooling function of the medical device according to the exemplary embodiment of the present disclosure. FIG. 9 illustrates the exemplary embodiment in which the operation method in FIG. 6 and the operation method in FIG. 7 are sequentially connected to each other. Accordingly, some steps in FIG. 9 may mean substantially the same as steps FIGS. 6 and 7, and in this case, the description of each step may be replaced with the description of FIG. 6 or FIG. 7. Here, the specific description thereof may be omitted.

In step S301, a cooling can is mounted in a cooling can holder.

In step S302, a first valve is opened, and a refrigerant in the cooling can moves into a refrigerant chamber through a first flow path.

In step S303, a first pressure value of a first pressure sensor, a second pressure value of a second pressure sensor, and/or a refrigerant level value of a level sensor are checked.

In step S304, it is checked whether the first pressure value is greater than or equal to a first reference value (e.g., a NoCan reference value).

When the first pressure value is less than the first reference value, the process proceeds to step S305 and outputs a cooling-can replacement alarm (i.e., a NoCan alarm) through a display or a speaker, so as to enable a user to replace the cooling can.

When the first pressure value is greater than or equal to the first reference value, the process proceeds to step S306 and check whether the first pressure value is greater than or equal to a second reference value (e.g., a heating reference value).

When the first pressure value is less than the second reference value, the present exemplary embodiment proceeds to step S307, and a control unit operates a heater to heat the cooling can.

When the first pressure value is greater than or equal to the second reference value, the present exemplary embodiment proceeds to step S308.

In step S308, it is checked whether a refrigerant level value (or a level value) is greater than or equal to a third reference value (e.g., a Low reference value).

When the refrigerant level value is greater than or equal to the third reference value, the present exemplary embodiment proceeds to step S309, and the corresponding remaining amount of the refrigerant is displayed on the display on the basis of the refrigerant level value.

In step S310, a second valve is opened in order to cool a treatment area or a handpiece, etc.

In step S311, the refrigerant is output to the handpiece through a second flow path according to a refrigerant output signal that is input from the control unit or the handpiece.

Meanwhile, referring back to step S308, when the refrigerant level value is less than the third reference value, the present exemplary embodiment proceeds to step S312 in order to determine whether a gas discharge condition is met.

In step S312, it is checked whether the second pressure value is greater than or equal to a fourth reference value (e.g., a discharge reference value).

When the second pressure value is greater than or equal to the fourth reference value, the present exemplary embodiment proceeds to step S313, so as to discharge the gas inside the refrigerant chamber to the outside of the device or output a gas discharge alarm, so as to enable a user to directly initiate a gas discharge operation.

When the second pressure value is less than the fourth reference value, the present exemplary embodiment proceeds to step S314, and outputs a refrigerant shortage alarm or a cooling-can replacement alarm through the display or speaker so as to enable the user to replace the cooling can.

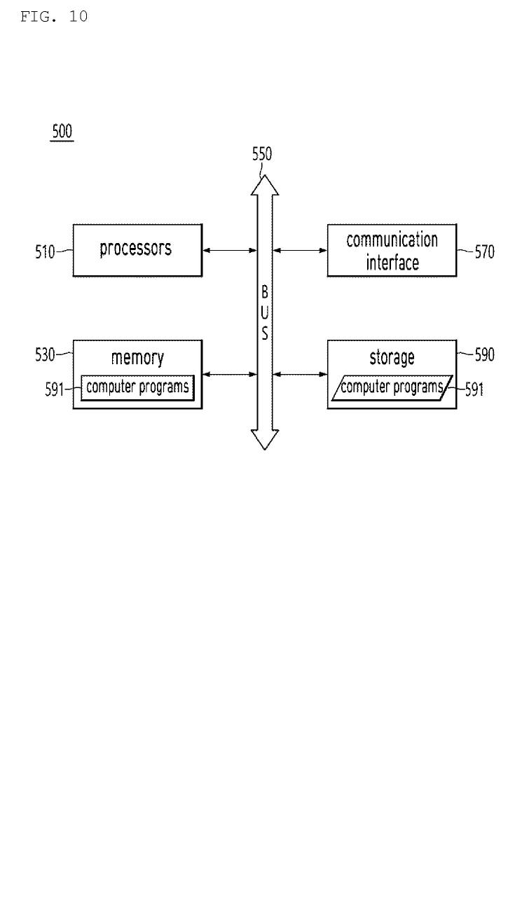

FIG. 10 is a flowchart illustrating an exemplary computing device in which an operation method of a medical system according to the exemplary embodiments of the present disclosure is implemented.

Hereinafter, the exemplary computing device 500 in which operating methods described in various exemplary embodiments of the present disclosure are implemented will be described with reference to FIG. 10. For example, the computing device 500 in FIG. 10 may be the medical device 1000 in FIG. 1.

FIG. 10 illustrates an exemplary hardware configuration diagram representing the computing device 500.

As illustrated in FIG. 10, the computing device 500 may include one or more processors 510, a bus 550, a communication interface 570, a memory 530 for loading computer programs 591 executed by the processors 510, and a storage 590 for storing the computer programs 591. However, only components related to the exemplary embodiments of the present disclosure are illustrated in FIG. 10. Accordingly, those skilled in the art to which the present disclosure belongs will appreciate that the present disclosure may further include general components other than those illustrated in FIG. 10.

The processors 510 control the overall operation of each component of the computing device 500. Each processor 510 may be configured to include at least one of a Central Processing Unit (CPU), a Micro Processor Unit (MPU), a Micro Controller Unit (MCU), a Graphics Processing Unit (GPU), or any type of processor well known in the technical field of the present disclosure. In addition, the processors 510 may perform operations for at least one of applications or programs for executing methods/operations according to various exemplary embodiments of the present disclosure. The computing device 500 may be provided with one or more processors.

The memory 530 stores various data, instructions, and/or information. The memory 530 may load one or more programs 591 from the storage 590 in order to execute the methods/operations according to various exemplary embodiments of the present disclosure. An example of the memory 530 may be a RAM, but is not limited thereto.

The bus 550 provides a communication function between the components of the computing device 500. The bus 550 may be implemented by applying various types of buses such as an address bus, a data bus, and a control bus.

The communication interface 570 supports wired and wireless Internet communication of the computing device 500. The communication interface 570 may also support various communication methods other than the Internet communication. To this end, the communication interface 570 may be configured to include a communication module well known in the technical field of the present disclosure.

The storage 590 may non-temporarily store one or more computer programs 591. The storage 590 may be configured to include non-volatile memory such as a Read Only Memory (ROM), an Erasable Programmable ROM (EPROM), an Electrically Erasable Programmable ROM (EEPROM), a flash memory, a hard disk, a removable disk, or any type of computer-readable recording medium well known in the technical field to which the present disclosure belongs.

The computer program 591 may include one or more instructions for which the methods/operations according to various exemplary embodiments of the present disclosure are implemented.

For example, the computer programs 591 may include the instructions for executing operations of: checking a first pressure value of a cooling can; checking a refrigerant level value of a refrigerant chamber when the first pressure value is greater than or equal to a first reference value and a second reference value; checking a second pressure value of the refrigerant chamber when the refrigerant level value is lower than a third reference value; and outputting a gas discharge alarm or initiating a discharge operation of a gas discharge unit when the refrigerant level value is lower than the third reference value and the second pressure value is higher than a fourth reference value.

In this case, the operation of checking of the first pressure value of the cooling can may include an operation of outputting an alarm indicating that the cooling can is not mounted in the cooling can holder when the first pressure value is lower than the first reference value, and operating the heater for heating the cooling can when the first pressure value is higher than the first reference value and lower than the second reference value.

In addition, the operation of checking the refrigerant level value of the refrigerant chamber may include an operation of displaying the remaining amount of the refrigerant on the display on the basis of the refrigerant level value when the refrigerant level value is higher than the third reference value.

In addition, the operation of checking the second pressure value of the refrigerant chamber may include an operation of outputting the refrigerant shortage alarm or the cooling-can replacement alarm when the refrigerant level value is lower than the third reference value and the second pressure value is lower than the fourth reference value.

In addition, the operation of outputting the gas discharge alarm or initiating the discharge operation of the gas discharge unit may include: the operation of displaying the gas discharge alarm on the display; the operation of closing the first valve connected to the cooling can and opening the third valve connected to the gas discharge unit; the operation of checking whether the pressure value of the refrigerant chamber is less than or equal to a predetermined value when the gas inside the refrigerant chamber is discharged through the gas discharge unit; the operation of closing the third valve when the pressure value of the refrigerant chamber is checked to be less than or equal to the predetermined value; and the operation of opening the first valve again.

When the computer programs 591 are loaded into the memory 530, the processors 510 may perform the methods/operations according to various exemplary embodiments of the present disclosure by executing the one or more instructions.

Although the exemplary embodiments of the present disclosure have been described above with reference to the accompanying drawings, it will be understood that those skilled in the art to which the present disclosure pertains may implement the present disclosure in other specific forms as well without departing from the technical spirit or essential features thereof. Therefore, the exemplary embodiments described above are to be understood in all respects as illustrative and not restrictive. The scope of protection of the present disclosure should be interpreted by the following claims, and all technical ideas within the scope equivalent thereto should be construed as being included in the scope of rights of the technical ideas defined by the present disclosure.

What is claimed is:

1. A medical device, comprising:
a cooling can holder held with a cooling can mounted therein;
a refrigerant chamber for accommodating a refrigerant introduced from the cooling can;
a refrigerant transfer unit for transferring the refrigerant accommodated in the refrigerant chamber toward a handpiece;
one or more sensors for measuring pressure or refrigerant levels of the refrigerant chamber; and
a control unit for outputting a gas discharge alarm or initiating a discharge operation of a gas discharge unit on the basis of values obtained by measuring pressure or refrigerant levels of the refrigerant chamber.

2. The medical device of claim 1, further comprising:
a first pressure sensor for measuring pressure of the cooling can,
wherein the control unit outputs an alarm indicating that a cooling can is not mounted in the cooling can holder when a first pressure value that is of the cooling can and measured by the first pressure sensor is lower than a first reference value.

3. The medical device of claim 2, wherein the control unit operates a heater for heating the cooling can when the first pressure value is higher than the first reference value and lower than a second reference value.

4. The medical device of claim 3, wherein the heater has an annular or curved shape at least partially surrounding the cooling can and is operated until a pressure of the cooling can reaches a predetermined pressure value or until a temperature of the cooling can reaches a predetermined temperature value.

5. The medical device of claim 1, wherein the one or more sensors comprises:
one or more level sensors for measuring refrigerant levels in the refrigerant chamber; and
a second pressure sensor for measuring pressure in the refrigerant chamber,
wherein the control unit outputs a gas discharge alarm or initiates a discharge operation of a gas discharge unit when a refrigerant level value that is of the refrigerant chamber and measured by the one or more level sensors is lower than a third reference value, and a second pressure value that is of the refrigerant chamber and measured by the second pressure sensor is higher than a fourth reference value.

6. The medical device of claim 5, wherein the one or more level sensors comprise:
a plurality of level sensors respectively corresponding to refrigerant levels different from each other of the refrigerant chamber, and
the control unit determines refrigerant levels of the refrigerant chamber on the basis of an “on” or “off” value of each of the plurality of level sensors, and determines that a refrigerant level value of the refrigerant chamber is lower than a third reference value when all the plurality of level sensors have “off” values.

7. The medical device of claim 1, wherein the one or more sensors comprise:
a second pressure sensor for measuring pressure of the refrigerant chamber, and
the control unit outputs agas discharge alarm or initiates adischarge operation of agas discharge unit when a second pressure value that is of the refrigerant chamber and measured by the second pressure sensor is higher than a fourth reference value.

8. The medical device of claim 6, wherein the control unit outputs a refrigerant shortage alarm or a cooling-can replacement alarm when a refrigerant level value of the refrigerant chamber is lower than the third reference value and the second pressure value is lower than a fourth reference value.

9. The medical device of claim 6, wherein the control unit displays a remaining amount of a refrigerant on a display on the basis of refrigerant levels that are of the refrigerant chamber and measured by the one or more level sensors.

10. The medical device of claim 1, wherein the gas discharge unit is connected to the refrigerant chamber through a third flow path, and
a high pressure protector capable of forcibly discharging gas inside the refrigerant chamber even in a state where the discharge operation of the gas discharge unit is not initiated is provided on a path of the third flow path.

11. The medical device of claim 1, further comprising:
a water hammer arrestor for mitigating a water hammer phenomenon due to refrigerant flow, and
the water hammer arrestor is coupled to a side surface of the refrigerant chamber.

12. An operation method of a medical device performed by a computing device, the operation method comprising:
checking a first pressure value of a cooling can;
checking a refrigerant level value of a refrigerant chamber when the first pressure value is greater than or equal to a first reference value and a second reference value;
checking a second pressure value of the refrigerant chamber when the refrigerant level value is lower than a third reference value; and
outputting a gas discharge alarm or initiating a discharge operation of a gas discharge unit when the refrigerant level value is lower than the third reference value and the second pressure value is higher than a fourth reference value.

13. The operation method of claim 12, wherein the checking of the first pressure value of the cooling can comprises:
outputting an alarm indicating that a cooling can is not mounted in the cooling can holder when the first pressure value is lower than the first reference value and operating a heater for heating the cooling can when the first pressure value is higher than the first reference value and lower than the second reference value.

14. The operation method of claim 12, wherein the checking of the refrigerant level value of the refrigerant chamber comprises:
displaying a remaining amount of refrigerant on a display on the basis of the refrigerant level value.

15. The operation method of claim 12, wherein the checking of the second pressure value of the refrigerant chamber comprises:
outputting a refrigerant shortage alarm or a cooling-can replacement alarm when the refrigerant level value is lower than the third reference value and the second pressure value is lower than the fourth reference value.

16. The operation method of claim 12, wherein the outputting of the gas discharge alarm or initiating of the discharge operation of the gas discharge unit comprises:
displaying the gas discharge alarm on the display;
closing a first valve connected to the cooling can and opening a third valve connected to the gas discharge unit;
checking whether a pressure value of the refrigerant chamber is less than or equal to a predetermined value when gas inside the refrigerant chamber is discharged through the gas discharge unit;
closing the third valve when a pressure value of the refrigerant chamber is checked to be less than or equal to a predetermined value; and
opening the first valve again.
